(19) United States
(12) Patent Application Publication
(10) Pub. No.: US 2023/0190956 A1
(43) Pub. Date: Jun. 22, 2023

(54) METHODS FOR PREVENTING INDUCTION OF IMMUNE RESPONSES TO THE TRANSDUCED CELLS EXPRESSING A TRANSGENE PRODUCT AFTER OCULAR GENE THERAPY

(71) Applicants: INSERM (Institut National de la Santé et de la Recherche Médicale), Paris (FR); Genethon, Evry (FR); Université d'Evry-Val-d'Essonne, Evry (FR)

(72) Inventor: Fisson SYLVAIN, Evry Cedex (FR)

(21) Appl. No.: 17/996,987

(22) PCT Filed: Apr. 23, 2021
(86) PCT No.: PCT/EP2021/060647

§ 371 Date: Oct. 24, 2022

(30) Foreign Application Priority Data
Apr. 24, 2020 (EP) 20315217.8
Apr. 27, 2020 (EP) 20305407.7

(51) Int. Cl. A61K 48/00; A61K 38/16; A61P 27/02

(52) U.S. Cl. CPC A61K 48/005; A61K 48/0083; A61K 48/0075; A61K 38/162; A61P 27/02; C12N 2750/14133; C12N 2750/14143

(57) ABSTRACT

Despite the eye's immune-privileged status, a secondary loss of vision in some patients treated with AAV led the inventors to question the immunogenicity of AAV vectors after a subretinal injection. The inventors thus characterized anti-transgene and anti-capsid immune responses induced in the periphery after the subretinal AAV injection. Different doses of AAV8 encoding reporter proteins fused with the HY male antigen were injected at day 0 into the subretinal space of adult immunocompetent C57BL/6 female mice. Subretinal AAV injection induced a dose-dependent proinflammatory immune response to the transgene product, correlated with local transgene expression. In order to trigger a subretinal-associated immune inhibition (SRAII) mechanism, some mice were co-injected subretinally at day 0 with AAV and HY peptides. Interestingly, this subretinal co-injection of AAV8 with peptides of the transgene product modulated the anti-transgene T-cell immune response, even at high dose of vector (5.10¹⁰ vg). This immunodulation was also confirmed in a pathophysiological murine model of retinal degeneration. The inventors also demonstrated that injection of AAV8 in the subretinal space induces proinflammatory peripheral immune responses to the transgene and the capsid that could be counteracted y co-injection with transgene peptides. Accordingly, the object of the present invention is to provide methods for preventing induction of immune responses to the transgene product and the AAV capsid after ocular gene therapy.

### FIELD OF THE INVENTION

The present invention is in the field of medicine and in particular gene therapy and ophthalmology.

### BACKGROUND OF THE INVENTION

In 1996, Ali et al. opened a new path toward adeno-associated virus (AAV)-mediated gene transfer in the retina by showing that photoreceptors and retinal pigment epithelium cells can be efficiently transduced by an AAV2 vector (Ali et al., 1996). During the following decade, several studies sought to characterize the tropism of several AAV serotypes in the retina (Allocca et al., 2007; Auricchio et al., 2001; Lebherz et al., 2008; Weber et al., 2003). Preclinical studies that were performed with AAV in non-human primates (Jacobson et al., 2006a; Maclachlan et al., 2011; Ramachandran et al., 2016; Vandenberghe et al., 2013) and dogs (Acland et al., 2001, 2005; Jacobson et al., 2006b; Le Meur et al., 2007; Petit et al., 2012) aimed at treating monogenic retinal dystrophies such as Leber's congenital amaurosis (LCA). In 2007, the first clinical trials for the correction of LCA by AAV-mediated ocular gene transfer began, led by Samuel Jacobson (NCT00481546), Robin Ali (NCT00643747) and Albert Maguire (NCT00516477). These three trials were soon followed by several others, for LCA (Timothy Stout NCT00749957, Michel Weber NCT01496040) and for other diseases such as choroideremia (NCT01461213) and age-related macular degeneration (NCT01024998, NCT01494805). Preliminary results published soon afterwards reported the safety of AAV vectors, along with vision improvement in some patients (Bainbridge et al., 2015; Hauswirth et al., 2008; Le Meur et al., 2017; Maguire et al., 2009).

Over the long term, however, some patients have experienced secondary loss of vision in the treated eye (Bainbridge et al., 2015; Jacobson et al., 2015). What can explain this phenomenon? Several possibly complementary hypotheses may be suggested. One is the ongoing degenerative process, which could lead to the programmed death of degenerating cells, despite the partial rescue of their function (Cideciyan et al., 2013). Another might be the induction of gene expression or post-transcriptional regulatory mechanisms, as described for Duchenne muscular dystrophy (Dupont et al., 2015). The third hypothesis involves the induction of immune responses to the transduced cells expressing the transgene product, a neo-antigen imported by the AAV vector, in addition to an immune response against vector capsid proteins.

The well-known immune privilege of the eye appears to have resulted in a failure to fully consider the role of immune response. Several properties of the eye limit and tightly control the induction of proinflammatory immune responses. Locally, physical barriers, such as the tight junctions that constitute the blood-retinal barrier, limit exchanges with the rest of the organism (Rizzolo et al., 2011). At the same time, the secretion of a large panel of anti-inflammatory molecules such as TGF-β (Stein-Streilein, 2013; Taylor et al., 1997) tends to inhibit immune responses. Moreover, immunomodulatory mechanisms can induce an antigen-specific immune deviation in the periphery after its introduction into the eye; that is, injection of antigen into the anterior chamber or subretinal space induces respectively anterior chamber-associated immune deviation (ACAID) (Vendomele et al., 2017) or subretinal-associated immune inhibition (SRAII) (Vendomele et al., 2018). Nonetheless, the eye is not hermetic to inflammatory processes. In several of its compartments, viruses and bacteria can induce inflammation such as endophthalmitis and uveitis (Chan et al., 2017; Kurniawan et al., 2017). Clinical trials of AAV-mediated ocular gene therapy appear to produce quite low levels of adaptive immune response, although ophthalmologic examinations have revealed transient and sometimes subclinical inflammation in several patients during the first few days (NCT00643747, NCT01494805). For obvious ethical reasons, immune monitoring has been performed only on blood samples, since in-depth investigation of the immunological mechanisms involved in these innovative approaches has not been possible.

### SUMMARY OF THE INVENTION

As defined by the claims, the present invention relates methods for preventing induction of immune responses to the transgene product and the AAV capsid after ocular gene therapy.

### DETAILED DESCRIPTION OF THE INVENTION

For a decade, AAV-mediated gene transfer has been tested in clinical trials to treat ocular diseases. Despite the eye's immune-privileged status, a secondary loss of vision in some patients treated with AAV led the inventors to question the immunogenicity of AAV vectors after a subretinal injection. The inventors thus aimed to characterize anti-transgene and anti-capsid immune responses induced in the periphery after the subretinal AAV injection. Different doses of AAV8 encoding reporter proteins (GFP or Luc2) fused with the HY male antigen were injected at day 0 into the subretinal space of adult immunocompetent C57BL/6 female mice. The transgene encoding the HY male antigen, contained MHC class I and MHC class II-restricted T cell epitopes (UTY and DBY peptides immuno-dominant in H-2^{b} female mice), and was packaged into AAV8 under PGK promoter. The mice were subcutaneously immunized at day 14 with or without HY peptides, and their T-cell immune responses in the spleen were analyzed at day 21 by an IFN-γ ELISpot assay after in vitro restimulation with HY peptides. Transgene expression was monitored over time with bioluminescence imaging and was correlated to the systemic anti-transgene (HY) T-cell immune responses (FIG. 1). Data showed that following the subretinal injection of AAV8, the level of transgene expression was correlated to the AAV injected dose and was maintained up to 20 days (FIGS. 1A and 1B). Subretinal AAV injection induced a dose-dependent proinflammatory immune response to the transgene product, correlated with local transgene expression (FIG. 1C). In order to trigger a subretinal-associated immune inhibition (SRAII) mechanism (Vendomèle et al., 2018), some mice were co-injected subretinally at day 0 with AAV and HY peptides. Interestingly, this subretinal co-injection of AAV8 with peptides of the transgene product modulated (at least 40% inhibition) the anti-transgene T-cell pro-inflammatory immune response (FIG. 2) and transgene-specific cell cytotoxicity in vivo (FIG. 3), even at high dose of vector (5.10¹⁰ vg).

With the aim to explore the anti-capsid T-cell response, splenocytes harvested at day 21 in the previous experiments were analyzed at day 21 by an IFN-γ ELISpot assay after in vitro restimulation with AAV8. Data showed that subretinal injection of AAV8 (5.10¹⁰ vg) induced an anti-AAV8 proinflammatory T-cell immune response (FIG. 4). Interestingly, subretinal co-injection of AAV8 with peptides of the transgene product allowed a bystander modulation (68.9% inhibition) of the anti-capsid T-cell immune response. Taken together, the data demonstrate that injection of AAV8 in the subretinal space induces proinflammatory peripheral immune responses to the transgene and the capsid that could be counteracted by co-injection with transgene peptides.

Accordingly, the object of the present invention is to provide methods for preventing induction of immune responses to the transgene product and the AAV capsid after ocular gene therapy.

Main Definitions:

As used herein, the term “patient” or “patient in need thereof”, is intended for a human or non-human mammal. Typically the patient is affected or likely to be affected with a retinal disease.

As used herein, the term “retina” is a common short-hand term for a highly-organized and complex multilayer structure of the visual system. The retina comprises at least five different kinds of neurons including photoreceptors, bipolar cells, horizontal cells, amacrine cells, and ganglions. The term “retina” includes the inner retinal layer, which is proximal to the vitreous of the eye, as well as the outer retinal layer, proximal to the choroid (i.e., the vascular/connective layer between the retina and the sclera of the eye), and the layers therebetween. Each of these layers comprises one or more cell portions or types that are involved directly or indirectly in processing of visual information. Beginning at the outermost layer and moving inward toward the vitreous, the retina comprises at least the following layers: The outermost layer of the retina is the retinal pigment epithelium (“RPE”) which provides vital metabolic support to other retinal layers but is not directly involved in encoding visual stimuli into neurological signals, and is not responsive to light. RPE cells are darkly pigmented and absorb stray photons that would otherwise contribute to light scatter within the eye. The next few layers of the retina relate to various cell bodies or portions, including those of the photoreceptor cells, i.e., rods for night vision and cones for day vision. Photoreceptors are the cells that receive light and transduce visual information signals for processing. Photoreceptors have a metabolic rate that is among the highest of any cells in the body. The metabolic needs of these cells are accommodated by having these cells located near the choroidal blood supply. The outer aspect of photoreceptors is a distinct layer called the outer segment. This layer contains photopigments which absorb light and convert it into electrical signals. The next layer of the retina is the inner segment of the photoreceptors, which contains many of the non-nuclear organelles of the photoreceptors. The outer limiting membrane (“OLM”), formed by interconnecting processes of retinal glial cells (aka Muller cells), separates the inner segment of the photoreceptor cells from their nuclei. The photoreceptor nuclei form the next distinct retinal layer, referred to as the outer nuclear layer (“ONL”). Continuing inward, the next retinal layer is the outer plexiform layer (“OPL”) which comprises the first layer of synaptic structures encountered, including dendrites of bipolar and horizontal layers, the synaptic endings of the photoreceptors, and other synapses. The inner nuclear layer (“INL”) is the next retinal layer, comprising bodies of the bipolar and horizontal cells, as well as the bodies of various types of amacrine cells. The next layer is the inner plexiform layer (“IPL”) comprising synapses of bipolar, horizontal, and amacrine cells. The innermost cell body layer is the ganglion cell layer (“GCL”) which is comprised of from about 80% parvo (or midget) cells, from about 10% parasol or macro cells, and other ganglion cells. The next layer of the retina, the nerve fiber layer (“NFL”) comprises the axons of the ganglion cells. These nerves are not myelinated within the eye, however they become so as they leave the eye to form the optic nerve. The innermost layer of the retina is the internal limiting membrane (“ILM”), which separates the retina from the vitreous humor.

As used herein, the term “retinal cell” can refer herein to any of the cell types that comprise the retina, such as retinal ganglion cells, amacrine cells, horizontal cells, bipolar cells, and photoreceptor cells including rods and cones, Muller glial cells, and retinal pigmented epithelium.

As used herein, the term “subretinal space” refers to the location in the retina between the photoreceptor cells and the retinal pigment epithelium cells. The subretinal space may be a potential space, such as prior to any subretinal injection of fluid. The subretinal space may also contain a fluid that is injected into the potential space. In this case, the fluid is “in contact with the subretinal space.” Cells that are “in contact with the subretinal space” include the cells that border the subretinal space, such as RPE and photoreceptor cells.

As used herein, the term “bleb” refers to a fluid space within the subretinal space of an eye. A bleb of the invention may be created by a single injection of fluid into a single space, by multiple injections of one or more fluids into the same space, or by multiple injections into multiple spaces, which when repositioned create a total fluid space useful for achieving a therapeutic effect over the desired portion of the subretinal space.

As used herein the term “retinal disease” refers to a broad class of diseases wherein the functioning of the retina is affected for example due to a damage or degeneration of the photoreceptors; ganglia or optic nerve; or even neovascularization. One skilled in the art can distinguish inherited retinal diseases and acquired retinal diseases. Representative examples of retinal acquired diseases include but are not limited to macular degeneration such as age related macular degeneration, and diabetic retinopathies. Examples of inherited retinal diseases include but are not limited to retinitis pigmentosa, Leber's congenital Amaurosis, X-linked Retinoschisis. Thus non-limiting examples of retinal diseases include: autosomal recessive severe early-onset retinal degeneration (Leber's Congenital Amaurosis), congenital achromatopsia, Stargardt's disease, Best's disease, Doyne's disease, cone dystrophy, retinitis pigmentosa, X-linked retinoschisis, Usher's syndrome, age related macular degeneration, atrophic age related macular degeneration, neovascular AMD, diabetic maculopathy, proliferative diabetic retinopathy (PDR), cystoid macular oedema, central serous retinopathy, retinal detachment, intra-ocular inflammation, glaucoma, posterior uveitis, choroideremia, and Leber hereditary optic neuropathy.

As used herein, the term “vision loss” refers to reduction in sight and includes partial and complete loss or reduction in sight. The term “secondary vision loss” denotes a vision loss that follows the ocular gene therapy after a while despite some clinical improvements observed in the earlier phases of treatment. Methods of assessing vision loss are known in the art, and include objective as well as subjective (e.g., subject reported) measures. For example, to measure the effectiveness of a treatment on a subject's visual function, one or more of the following may be evaluated: the subject's subjective quality of vision or improved central vision function (e.g., an improvement in the subject's ability to read fluently and recognize faces), the subject's visual mobility (e.g., a decrease in time needed to navigate a maze), visual acuity (e.g., an improvement in the subject's Log MAR score), microperimetry (e.g., an improvement in the subject's dB score), dark-adapted perimetry (e.g., an improvement in the subject's dB score), fine matrix mapping (e.g., an improvement in the subject's dB score), Goldmann perimetry (e.g., a reduced size of scotomatous area (i.e. areas of blindness) and improvement of the ability to resolve smaller targets), flicker sensitivities (e.g., an improvement in Hertz), autofluorescence, and electrophysiology measurements (e.g., improvement in ERG).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “gene therapy” refers to the introduction of a polynucleotide into a cell's genome that restores, corrects, or modifies the gene and/or expression of the gene. Thus the term “ocular gene therapy” refers to a gene therapy that is applied to the ocular sphere, in particular for expressing a transgene product in a retinal cell.

As used herein, the terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “derived from” refers to a process whereby a first component (e.g., a first polypeptide), or information from that first component, is used to isolate, derive or make a different second component (e.g., a second polypeptide that is different from the first one).

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the term “transgene” refers to a polynucleotide that is introduced into the cells of a tissue or an organ and is capable of being expressed under appropriate conditions, or otherwise conferring a beneficial property to the cells. A transgene is selected based upon a desired therapeutic outcome.

As used herein, the term “transgene product” refers to any molecule that is encoded by a transgene and confers a beneficial property to the cells or a desired therapeutic outcome. Typically, the transgene product is a polypeptide.

As used herein, the term “therapeutic level” refers to the amount of a transgene product or the level of activity of a transgene product sufficient to confer its therapeutic or beneficial effect(s) in the host receiving the transgene. Expression levels of the transgene or the levels of activity of the transgene product can be measured at the protein or the mRNA level using methods known in the art.

As used herein, the term “vector” refers to an agent capable of delivering and expressing the transgene in a host cell. The vector may be extrachromosomal (e.g. episome) or integrating (for being incorporated into the host chromosomes), autonomously replicating or not, multi or low copy, double-stranded or single-stranded, naked or complexed with other molecules (e.g. vectors complexed with lipids or polymers to form particulate structures such as liposomes, lipoplexes or nanoparticles, vectors packaged in a viral capsid, and vectors immobilised onto solid phase particles, etc.). The definition of the term “vector” also encompasses vectors that have been modified to allow preferential targeting to a particular host cell. A characteristic feature of targeted vectors is the presence at their surface of a ligand capable of recognizing and binding to a cellular and surface-exposed component such as a cell-specific marker, a tissue-specific marker or a cell-specific marker.

As used herein, the term “viral vector” encompasses vector DNA as well as viral particles generated thereof. Viral vectors can be replication-competent, or can be genetically disabled so as to be replication-defective or replication-impaired. The term “replication-competent” as used herein encompasses replication-selective and conditionally-replicative viral vectors which are engineered to replicate better or selectively in specific host cells (e.g. tumoral cells).

As used herein, the term “AAV” has its general meaning in the art and refers to adeno-associated virus, and may be used to refer to the virus itself or derivatives thereof. The term covers all serotypes and variants both naturally occurring and engineered forms. The term “AAV” includes but is not limited to AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), and AAV type 8 (AAV-8).) and AAV type 9 (AAV9). The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_002077 (AAV-1), AF063497 (AAV-1), NC_001401 (AAV-2), AF043303 (AAV-2), NC_001729 (AAV-3), NC_001829 (AAV-4), U89790 (AAV-4), NC_006152 (AAV-5), AF513851 (AAV-7), AF513852 (AAV-8), and NC_006261 (AAV-8).

As used herein, the term “rAAV” refers to recombinant adeno-associated virus, also referred to as a recombinant AAV vector (or “rAAV vector”). The term thus refers to an AAV vector comprising the transgene of interest for the genetic transformation of a cell. In general, the rAAV vectors contain 5′ and 3′ adeno-associated virus inverted terminal repeats (ITRs), and the transgene of interest operatively linked to sequences which regulate its expression in a target cell.

As used herein, the term “pseudotyped AAV vector” refers to a vector particle comprising a native AAV capsid including an rAAV vector genome and AAV Rep proteins, wherein Cap, Rep and the ITRs of the vector genome come from at least 2 different AAV serotypes.

As used herein, the term “capsid” refers to the protein coat of the virus or viral vector. The capsid of AAV (e.g., AAV2, AAVrh8R, etc.) is known to include three capsid proteins: VP1, VP2, and VP3. These proteins contain significant amounts of overlapping amino acid sequence and unique N-terminal sequences. An AAV2 capsid includes 60 subunits arranged by icoshedral symmetry (Xie, Q., et al. (2002) Proc. Natl. Acad. Sci. 99(16):10405-10). VP1, VP2, and VP3 have been found to be present in a 1:1:10 ratio.

As used herein, the term “non-viral vector” notably refers to a vector of plasmid origin, and optionally such a vector combined with one or more substances improving the transfectional efficiency and/or the stability of said vector and/or the protection of said vector.

As used herein, the term “transduced cell” relates to a genetically modified cell i.e. a cell wherein the transgene has been introduced deliberately. The herein provided transduced cell comprises the transgene of the present invention.

As used herein, the term “immune response” refers to a reaction of the immune system to an antigen in the body of a host, which includes generation of an antigen-specific antibody and/or cellular cytotoxic response. The immune response to an initial antigenic exposure (primary immune response) is typically, detectable after a lag period of from several days to two weeks; the immune response to subsequent stimulus (secondary immune response) by the same antigen is more rapid than in the case of the primary immune response. An immune response to a transgene product may include both humoral (e.g., antibody response) and cellular (e.g., cytolytic T cell response) immune responses that may be elicited to an immunogenic product encoded by the transgene. The level of the immune response can be measured by methods known in the art (e.g., by measuring antibody titer).

As used herein, term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR). The DNA targeting endonuclease can be a naturally occurring endonuclease (e.g., a bacterial meganuclease) or it can be artificially generated (e.g., engineered meganucleases, TALENs, or ZFNs, among others).

As used herein, the term “TALEN” has its general meaning in the art and refers to a transcription activator-like effector nuclease, an artificial nuclease which can be used to edit a target gene.

As used herein, the term “ZFN” or “Zinc Finger Nuclease” has its general meaning in the art and refers to a zinc finger nuclease, an artificial nuclease which can be used to edit a target gene.

As used herein, the term “CRISPR-associated endonuclease” has its general meaning in the art and refers to clustered regularly interspaced short palindromic repeats associated which are the segments of prokaryotic DNA containing short repetitions of base sequences.

The term “immunodominant peptide” is used herein to refer to a peptide that contains a T cell epitope that derives from the vector or the transgene product and that can thus induce a immune response (humoral and/or cell mediated response).

As used herein, the term “antigen-presenting cell” or “APC” refers to a class of cells capable of presenting antigen to T lymphocytes which recognize antigen when it is associated with a major histocompatibility complex molecule.

Methods:

The first object of the present invention relates to a method for preventing a secondary vision loss in a patient who received an ocular gene therapy with a vector containing a transgene comprising administering at least one peptide that derives from the transgene product or the vector, simultaneously to gene therapy thereby preventing induction of immune responses to the transduced cells expressing the transgene product.

In a more particular embodiment, the present invention relates to a method for preventing a secondary vision loss in a patient who received an ocular gene therapy with a vector containing a transgene comprising administering at least one peptide that derives from the transgene product or the vector, simultaneously to gene therapy thereby preventing induction of the cellular cytotoxic response to the transduced cells expressing the transgene product.

A further object of the present invention relates to a method for expressing a transgene of interest in the retina of a patient comprising the step consisting of injecting into the subretinal space an amount of a vector containing the transgene of interest in combination with an amount of at least one peptide that derives from the transgene product or the vector.

The methods of the present invention are particularly relevant for expressing a transgene of interest in the outer retina (photoreceptors and retinal pigment epithelium).

Accordingly, the present invention provides methods for treating a retinal disease in a patient in need thereof, comprising the general step of injecting into subretinal space an amount of a vector containing the transgene of interest in combination with an amount of at least one peptide that derives from the transgene product or the vector.

A wide variety of diseases of the eye may thus be treated given the teachings provided herein.

For example, the method of the invention is performed in order to treat or prevent macular degeneration. Briefly, the leading cause of visual loss in the elderly is macular degeneration (MD), which has an increasingly important social and economic impact in the United States. As the size of the elderly population increases in this country, age related macular degeneration (AMD) will become a more prevalent cause of blindness than both diabetic retinopathy and glaucoma combined. Although laser treatment has been shown to reduce the risk of extensive macular scarring from the “wet” or neovascular form of the disease, there are currently no effective treatments for the vast majority of patients with MD.

The method of the invention may also be performed in order to treat or prevent an inherited retinal degeneration. One of the most common inherited retinal degenerations is retinitis pigmentosa (RP), which results in the degeneration of photoreceptor cells, and the RPE. Other inherited conditions include Bardet-Biedl syndrome (autosomal recessive); Bassen-Kornzweig syndrome, Best disease, choroidema, gyrate atrophy, Leber congenital amaurosis, Refsun syndrome, Stargardt disease; Cone or cone-rod dystrophy (autosomal dominant and X-linked forms); Congenital stationary night blindness (autosomal dominant, autosomal recessive and X-linked forms); Macular degeneration (autosomal dominant and autosomal recessive forms); Optic atrophy, autosomal dominant and X-linked forms); Retinitis pigmentosa (autosomal dominant, autosomal recessive and X-linked forms); Syndromic or systemic retinopathy (autosomal dominant, autosomal recessive and X-linked forms); and Usher syndrme (autosomal recessive).

One skilled in the art knows, by its knowledge of the scientific literature in his field, which are the transgenes that may be more appropriate to treat a specific retinal disease.

In some embodiments, the transgene product is a polypeptide that will enhance the function of a retinal cell, e.g., the function of a rod or cone photoreceptor cell, a retinal ganglion cell, a Müller cell, a bipolar cell, an amacrine cell, a horizontal cell, or a retinal pigmented epithelial cell. Examples of polynucleotides of interest include but are not limited to those encoding for a polypeptide selected from the group consisting of neuroprotective polypeptides (e.g., GDNF, CNTF, NT4, NGF, and NTN); anti-angiogenic polypeptides (e.g., a soluble vascular endothelial growth factor (VEGF) receptor; a VEGF-binding antibody; a VEGF-binding antibody fragment (e.g., a single chain anti-VEGF antibody); endostatin; tumstatin; angiostatin; a soluble Fit polypeptide (Lai et al. (2005) Mol. Ther. 12:659); an Fc fusion protein comprising a soluble Fit polypeptide (see, e.g., Pechan et al. (2009) Gene Ther. 16: 10); pigment epithelium-derived factor (PEDF); a soluble Tie-2 receptor; etc.); tissue inhibitor of metalloproteinases-3 (TIMP-3); a light-responsive opsin, e.g., a rhodopsin; anti-apoptotic polypeptides (e.g., Bcl-2, Bcl-X1); and the like. Other suitable polypeptides include, but are not limited to, glial derived neurotrophic factor (GDNF); fibroblast growth factor 2; neurturin (NTN); ciliary neurotrophic factor (CNTF); nerve growth factor (NGF); neurotrophin-4 (NT4); brain derived neurotrophic factor (BDNF); epidermal growth factor; rhodopsin; X-linked inhibitor of apoptosis; and Sonic hedgehog. Suitable light-responsive opsins include, e.g., a light-responsive opsin as described in U.S. Patent Publication No. 2007/0261127 (e.g., ChR2; Chop2, CaTCh); U.S. Patent Publication No. 2001/0086421; U.S. Patent Publication No. 2010/0015095; and Diester et al. (2011) Nat. Neurosci. 14:387.14:387 or halorhodopsin (e.g. eNpHR) or other light gated ion channel or proton pumps. Suitable polypeptides also include retinoschisin. Suitable polypeptides include, e.g., retinitis pigmentosa GTPase regulator (RGPR)-interacting protein-1 (see, e.g., GenBank Accession Nos. Q96KN7, Q9EPQ2, and Q9GLM3); peripherin-2 (Prph2) (see, e.g., GenBank Accession No. NP_000313; peripherin; a retinal proteinisomerase (RPE65), (see, e.g., GenBank AAC39660; and Morimura et al. (01998) Proc. Natl. Acad. Sci. USA 95:3088); and the like. Suitable polypeptides also include: CHM (choroidermia (Rab escort protein 1)), a polypeptide that, when defective or missing, causes choroideremia (see, e.g., Donnelly et al. (1994) Hum. Mol. Genet. 3: 1017; and van Bokhoven et al. (1994) Hum. Mol. Genet. 3: 1041); and Crumbs homolog 1 (CRB1), a polypeptide that, when defective or missing, causes Leber congenital amaurosis and retinitis pigmentosa (see, e.g., den Hollander et al. (1999) Nat. Genet. 23:217; and GenBank Accession No. CAM23328). Suitable polypeptides also include polypeptides that, when defective or missing, lead to achromotopsia, where such polypeptides include, e.g., cone photoreceptor cGMP-gated channel subunit alpha (CNGA3) (see, e.g., GenBank Accession No. NP 001289; and Booij et al. (2011) Ophthalmology 118: 160-167); cone photoreceptor cGMP-gated cation channel beta-subunit (CNGB3) (see, e.g., Kohl et al.(2005) Eur J Hum Genet. 13(3):302); guanine nucleotide binding protein (G protein), alpha transducing activity polypeptide 2 (GNAT2) (ACHM4); and ACHM5; and polypeptides that, when defective or lacking, lead to various forms of color blindness (e.g., L-opsin, M-opsin, and S-opsin). See Mancuso et al. (2009) Nature 461(7265):784-787. In a particular embodiment, the transgene of interest may encode for a neurotrophic factor. As used herein, the “neurotrophic factor” is a generic term of proteins having a physiological action such as survival and maintenance of nerve cells, promotion of neuronal differentiation. Examples of neurotrophic factors include but are not limited to bFGF, aFGF, BDNF, CNTF, IL-lbeta, NT-3, IGF-II, GDNF, NGF and RdCVF.

In some embodiments, the transgene product of interest is an endonuclease that provides for site-specific knock-down of gene function, e.g., where the endonuclease knocks out an allele associated with a retinal disease. For example, where a dominant allele encodes a defective copy of a gene that, when wild-type, is a retinal structural protein and/or provides for normal retinal function, a site-specific endonuclease can be targeted to the defective allele and knock out the defective allele. In addition to knocking out a defective allele, a site-specific nuclease can also be used to stimulate homologous recombination with a donor DNA that encodes a functional copy of the protein encoded by the defective allele. Thus, e.g., the method of the invention can be used to deliver both a site-specific endonuclease that knocks out a defective allele, and can be used to deliver a functional copy of the defective allele, resulting in repair of the defective allele, thereby providing for production of a functional retinal protein (e.g., functional retinoschisin, functional RPE65, functional peripherin, etc.). See, e.g., Li et al. (2011) Nature 475:217.

In some embodiments, the DNA targeting endonuclease of the present invention is a TALEN. TALENs are produced artificially by fusing a TAL effector (“TALE”) DNA binding domain, e.g., one or more TALEs, e.g., 1, 2, 3, 4, 5, 6, 7, 8, 9 or 10 TALEs to a DNA-modifying domain, e.g., a FokI nuclease domain. Transcription activator-like effects (TALEs) can be engineered to bind any desired DNA sequence (Zhang (2011), Nature Biotech. 29: 149-153).

By combining an engineered TALE with a DNA cleavage domain, a restriction enzyme can be produced which is specific to any desired DNA sequence. These can then be introduced into a cell, wherein they can be used for genome editing (Boch (2011) Nature Biotech. 29: 135-6; and Boch et al. (2009) Science 326: 1509-12; Moscou et al. (2009) Science 326: 3501). TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence (Zhang (2011), Nature Biotech. 29: 149-153). To produce a TALEN, a TALE protein is fused to a nuclease (N), e.g., a wild-type or mutated FokI endonuclease. Several mutations to FokI have been made for its use in TALENs; these, for example, improve cleavage specificity or activity (Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96). The Fold domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity (Miller et al. (2011) Nature Biotech. 29: 143-8). TALEN can be used inside a cell to produce a double-strand break in a target nucleic acid, e.g., a site within a gene. A mutation can be introduced at the break site if the repair mechanisms improperly repair the break via non-homologous end joining (Huertas, P., Nat. Struct. Mol. Biol. (2010) 17: 11-16). For example, improper repair may introduce a frame shift mutation. Alternatively, foreign DNA can be introduced into the cell along with the TALEN; depending on the sequences of the foreign DNA and chromosomal sequence, this process can be used to modify a target gene via the homologous direct repair pathway, e.g., correct a defect in the target gene, thus causing expression of a repaired target gene, or e.g., introduce such a defect into a wt gene, thus decreasing expression of a target gene.

In some embodiments, the DNA targeting endonuclease of the present invention is a ZFN. Like a TALEN, a ZFN comprises a DNA-modifying domain, e.g., a nuclease domain, e.g., a Fold nuclease domain (or derivative thereof) fused to a DNA-binding domain. In the case of a ZFN, the DNA-binding domain comprises one or more zinc fingers, e.g., 1, 2, 3, 4, 5, 6, 7, 8, 9 or 10 zinc fingers (Carroll et al. (2011) Genetics Society of America 188: 773-782; and Kim et al. (1996) Proc. Natl. Acad. Sci. USA 93: 1156-1160). A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art (Sera (2002), Biochemistry, 41:7074-7081; Liu (2008) Bioinformatics, 24:1850-1857). A ZFN using a FokI nuclease domain or other dimeric nuclease domain functions as a dimer. Thus, a pair of ZFNs are required to target non-palindromic DNA sites. The two individual ZFNs must bind opposite strands of the DNA with their nucleases properly spaced apart (Bitinaite et al. (1998) Proc. Natl. Acad. Sci. USA 95: 10570-5). Also like a TALEN, a ZFN can create a DSB in the DNA, which can create a frame-shift mutation if improperly repaired, e.g., via non-homologous end joining, leading to a decrease in the expression of a target gene in a cell.

In some embodiments, the DNA targeting endonuclease of the present invention is a CRISPR-associated endonuclease. In bacteria the CRISPR/Cas loci encode RNA-guided adaptive immune systems against mobile genetic elements (viruses, transposable elements and conjugative plasmids). Three types (I-VI) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonucleases Cas9 and Cpfl belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3^{rd} or the 4^{th} nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector.

In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as pX330, pX260 or pMJ920 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, Mass.).

In some embodiments, the CRISPR-associated endonuclease is a Cpfl nuclease. As used herein, the term “Cpfl protein” to a Cpfl wild-type protein derived from Type V CRISPR-Cpfl systems, modifications of Cpfl proteins, variants of Cpfl proteins, Cpfl orthologs, and combinations thereof. The cpfl gene encodes a protein, Cpfl, that has a RuvC-like nuclease domain that is homologous to the respective domain of Cas9, but lacks the HNH nuclease domain that is present in Cas9 proteins. Type V systems have been identified in several bacteria, including Parcubacteria bacterium GWC2011_GWC2_44_17 (PbCpfl), Lachnospiraceae bacterium MC2017 (Lb3 Cpfl), Butyrivibrio proteoclasticus (BpCpfl), Peregrinibacteria bacterium GW2011_GWA 33_10 (PeCpfl), Acidaminococcus spp. BV3L6 (AsCpfl), Porphyromonas macacae (PmCpfl), Lachnospiraceae bacterium ND2006 (LbCpfl), Porphyromonas crevioricanis (PcCpfl), Prevotella disiens (PdCpfl), Moraxella bovoculi 237(MbCpfl), Smithella spp. SC_K08D17 (SsCpfl), Leptospira inadai (LiCpfl), Lachnospiraceae bacterium MA2020 (Lb2Cpf1), Franciscella novicida U112 (FnCpfl), Candidatus methanoplasma termitum (CMtCpfl), and Eubacterium eligens (EeCpfl). Recently it has been demonstrated that Cpfl also has RNase activity and it is responsible for pre-crRNA processing (Fonfara, I., et al., “The CRISPR-associated DNA-cleaving enzyme Cpfl also processes precursor CRISPR RNA,” Nature 28; 532(7600):517-21 (2016)).

In some embodiments, the transgene product is an interfering RNA (RNAi). Typically, suitable RNAi include RNAi that decrease the level of an apoptotic or angiogenic factor in a cell. For example, an RNAi can be a shRNA or siRNA that reduces the level of a transgene product that induces or promotes apoptosis in a cell. Genes whose transgene products induce or promote apoptosis are referred to herein as “pro-apoptotic genes” and the products of those genes (mRNA; protein) are referred to as “pro-apoptotic transgene products.” Pro-apoptotic transgene products include, e.g., Bax, Bid, Bak, and Bad transgene products. See, e.g., U.S. Pat. No. 7,846,730. Interfering RNAs could also be against an angiogenic product, for example VEGF (e.g., Cand5; see, e.g., U.S. Patent Publication No. 2011/0143400; U.S. Patent Publication No. 2008/0188437; and Reich et al. (2003) Mol. Vis. 9:210), VEGFR1 (e.g., Sirna-027; see, e.g., Kaiser et al. (2010) Am. J. Ophthalmol. 150:33; and Shen et al. (2006) Gene Ther. 13:225), or VEGFR2 (Kou et al. (2005) Biochem. 44: 15064). See also, U.S. Pat. Nos. 6,649,596, 6,399,586, 5,661,135, 5,639,872, and 5,639,736; and U.S. Pat. Nos. 7,947,659 and 7,919,473.

In some embodiments, the vector containing the transgene of interest is selected from the group consisting of viral and non-viral vectors.

Typically viral vectors include, but are not limited to nucleic acid sequences from the following viruses: RNA viruses such as a retrovirus (as for example moloney murine leukemia virus and lentiviral derived vectors), harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus and AAV vectors. Preferred viral gene delivery vector are rAAV vectors.

In some embodiments, the AAV vector is an AAV8 vector.

In some embodiments, the viral vector is a pseudotyped AAV vector. Examples of AAV chimeric vectors include but are not limited to AAV2/5, AAV2/6, and AAV2/8. In some embodiments, the AAV chimeric vector is the AAV2/8 described in U.S. Pat. No. 7,282,199, which is incorporated by reference herein.

In some embodiments, the viral vector is an engineered AAV vector. In particular, the engineered AAVvector is the SH10 vector as described in Klimczak R R, Koerber J T, Dalkara D, Flannery J G, Schaffer D V. 2009. A novel adeno-associated viral variant for efficient and selective intravitreal transduction of rat Muller cells. PLoS One 4(10):e7467. AAV variant ShH10 is closely related to AAV serotype 6 (AAV6). In some embodiments, the AAV engineered vector has a mutated capsid, in particular a tyrosine mutated capsid. In some embodiments, the AAV engineered vector is the one described in WO2012145601 which is incorporated by reference herein. In some embodiments, the vector is a recombinant adeno-associated virus (rAAV) virion comprising a variant AAV capsid protein, wherein the variant AAV capsid protein comprises an insertion of from about 5 amino acids to about 11 amino acids in the capsid protein GH loop relative to a corresponding parental AAV capsid protein, and wherein the variant capsid protein confers increased infectivity of a retinal cell compared to the infectivity of the retinal cell by an AAV virion comprising the corresponding parental AAV capsid protein. In some embodiments, the vector is the AAV2-7m8 as described in WO2012145601 and Dalkara D, Byrne L C, Klimczak R R, Visel M, Yin L, Merigan W H, Flannery J G, Schaffer D V. In vivo-directed evolution of a new adeno-associated virus for therapeutic outer retinal gene delivery from the vitreous. Sci Transl Med. 2013 Jun. 12;5(189):189ra76. Other examples include those described in:
Kay C N, Ryals R C, Aslanidi G V, Min S H, Ruan Q, Sun J, Dyka F M, Kasuga D, Ayala A E, Van Vliet K, Agbandje-McKenna M, Hauswirth W W, Boye S L, Boye S E. Targeting photoreceptors via intravitreal delivery using novel, capsid-mutated AAV vectors. PLoS One. 2013 Apr. 26;8(4):e62097. doi: 10.1371/journal.pone.0062097.
Dalkara D, Byrne L C, Lee T, Hoffmann N V, Schaffer D V, Flannery J G. Enhanced gene delivery to the neonatal retina through systemic administration of tyrosine-mutated AAV9. Gene Ther. 2012 February;19(2):176-81. doi: 10.1038/gt.2011.163. Epub 2011 Oct. 20.
Petrs-Silva H, Dinculescu A, Li Q, Min S H, Chiodo V, Pang J J, Zhong L, Zolotukhin S, Srivastava A, Lewin A S, Hauswirth W W. High-efficiency transduction of the mouse retina by tyrosine-mutant AAV serotype vectors. Mol Ther. 2009 March;17(3):463-71.
Petrs-Silva H, Dinculescu A, Li Q, Deng W T, Pang J J, Min S H, Chiodo V, Neeley A W, Govindasamy L, Bennett A, Agbandje-McKenna M, Zhong L, Li B, Jayandharan G R, Srivastava A, Lewin A S, Hauswirth W W. Novel properties of tyrosine-mutant AAV2 vectors in the mouse retina. Mol Ther. 2011 February;19(2):293-301. doi: 10.1038/mt.2010.234. Epub 2010 Nov. 2.

Non-viral vectors are widely documented in the literature which is accessible to persons skilled in the art (see for example Feigner et al., 1987, Proc. West. Pharmacol. Soc. 32, 115-121; Hodgson and Solaiman, 1996, Nature Biotechnology 14, 339-342; Remy et al., 1994, Bioconjugate Chemistry 5, 647-654). By way of illustration but without limitation, they may be polymers, lipids, in particular cationic lipids, liposomes, nuclear proteins or neutral lipids. These substances may be used alone or in combination. A combination which may be envisaged is a plasmid recombinant vector combined with cationic lipids (DOGS, DC-CHOL, spermine-chol, spermidine-chol and the like) and neutral lipids (DOPE). The choice of the plasmids which can be used in the context of the present invention is vast. They may be cloning and/or expression vectors. In general, they are known to a person skilled in the art and a number of them are commercially available, but it is also possible to construct them or to modify them by genetic engineering techniques. There may be mentioned, by way of examples, the plasmids derived from pBR322 (Gibco BRL), pUC (Gibco BRL), pBluescript (Stratagene), pREP4, pCEP4 (Invitrogene) or p Poly (Lathe et al., 1987, Gene 57, 193-201). Preferably, a plasmid used in the context of the present invention contains a replication origin ensuring the initiation of replication in a producing cell and/or a host cell (for example, the ColE1 origin may be selected for a plasmid intended to be produced in E. coli and the oriP/EBNA1 system may be selected if it is desired for it to be self-replicating in a mammalian host cell, Lupton and Levine, 1985, Mol. Cell. Biol. 5, 2533-2542; Yates et al., Nature 313, 812-815). It may comprise additional elements improving its maintenance and/or its stability in a given cell (cer sequence which promotes the monomeric maintenance of a plasmid (Summers and Sherrat, 1984, Cell 36, 1097-1103, sequences for integration into the cell genome).

In some embodiments, the vector may also comprise regulatory sequences allowing expression and, secretion of the encoded protein, such as e.g., a promoter, enhancer, polyadenylation signal, internal ribosome entry sites (IRES), sequences encoding protein transduction domains (PTD), and the like. In this regard, the vector comprises a promoter region, operably linked to the transgene of interest, to cause or improve expression of the protein in infected cells. Such a promoter may be ubiquitous, tissue-specific, strong, weak, regulated, chimeric, inducible, etc., to allow efficient and suitable production of the protein in the infected tissue. The promoter may be homologous to the encoded protein, or heterologous, including cellular, viral, fungal, plant or synthetic promoters. Most preferred promoters for use in the present invention shall be functional in cells or the retina, more preferably in photoreceptor or ganglion cells of the retina or in cells of the RPE. Examples of such regulated promoters include, without limitation, Tet on/off element-containing promoters, rapamycin-inducible promoters and metallothionein promoters. Examples of ubiquitous promoters include viral promoters, particularly the CMV promoter, the RSV promoter, the SV40 promoter, etc. and cellular promoters such as the PGK (phosphoglycerate kinase) promoter. The promoters may also be neurospecific promoters such as the Synapsin or the NSE (Neuron Specific Enolase) promoters (or NRSE (Neuron restrictive silencer element) sequences placed upstream from the ubiquitous PGK promoter), or promoters specific for various retinal cell types such as the RPE65, the VMD2, the Rhodopsin or the cone arrestin promoters. The vector may also comprise target sequences for miRNAs achieving suppression of transgene expression in non-desired cells. For example, suppression of expression in the hematopoietic lineages (“de-targeting”) enables stable gene transfer in the transduced cells by reducing the incidence and the extent of the transgene-specific immune response (Brown B D, Nature Medicine 2008). In a particular embodiment, the vector comprises a leader sequence allowing secretion of the encoded protein. Fusion of the transgene of interest with a sequence encoding a secretion signal peptide (usually located at the N-terminal end of secreted polypeptides) will allow the production of the therapeutic protein in a form that can be secreted from the transduced cells. Examples of such signal peptides include the albumin, the β-glucuronidase, the alkaline protease or the fibronectin secretory signal peptides. In a most preferred embodiment, the promoter is specific or functional in cells of the retina, in particular in photoreceptor or ganglion cells of the retina or in the RPE, i.e., allows (preferential) expression of the transgene in said cells. For example, suitable photoreceptor-specific regulatory elements include, e.g., a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9: 1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225).

In some embodiments, the peptide is an immunodominant peptide that derives from the transgene product or vector.

In some embodiments, an immunodominant peptide is selected for its ability to be presented by an antigen-presenting cell (APCs). APCs elicit a T cell response to a specific antigen by processing the antigen into a form that is capable of associating with a major histocompatibility complex molecule (MHC) on the surface of the APC. Major histocompatibility complex (MHC) class I and class II molecules play indeed a pivotal role in the adaptive branch of the immune system. Immunogenic peptide—MHC class I (pMHCI) complexes are presented on nucleated cells and are recognized by cytotoxic CD8+ T cells. The presentation of pMHCII by antigen-presenting cells [e.g., dendritic cells (DCs), macrophages, or B cells], on the other hand, can activate CD4+ T cells, leading to the coordination and regulation of effector cells. In all cases, it is a clonotypic T cell receptor that interacts with a given pMHC complex, potentially leading to sustained cell:cell contact formation and T cell activation. Thus, in some embodiments, the immunodominant peptide comprises a MHC-class I restricted epitope and/or a MHC-class II restricted epitope. In some embodiments, the immunodominant peptide comprises both a MHC-class I restricted epitope and a MHC-class II restricted epitope.

In sme embodiments, the immunodominant peptide derives from the capsid protein of the viral vector. In some embodiments, the immunodominant peptide derives from the VP1, VP2, or VP3 capsid protein the AAV vector (e.g. AAV8 vector).

In some embodiments, the immunodominant peptide derives from the transgene product.

In some embodiments, the vector is injected in the subretinal space simultaneously with 2, 3, 4, 5, 6, 8, 9 or 10 immunodominant peptides.

In some embodiments, the vector is injected with at least one immunodominant peptide comprising a MHC-class I restricted epitope and at least one immunodominant peptide comprising a MHC-class II restricted epitope.

Methods for identifying and characterizing immunodominant peptide are well known in the art. Typically, said methods include but are not limited to epitope prediction algorithms (Vita, Randi, et al. “The immune epitope database (IEDB) 3.0.” Nucleic acids research 43.D1 (2015): D405-D412; Jorgensen, Kasper W., et al. “Net MHC stab-predicting stability of peptide-MHC-I complexes; impacts for cytotoxic T lymphocyte epitope discovery.” Immunology 141.1 (2014): 18-26; Trolle, Thomas, et al. “Automated benchmarking of peptide-MHC class I binding predictions.” Bioinformatics 31.13 (2015): 2174-2181; Rammensee, H-G., et al. “SYFPEITHI: database for MHC ligands and peptide motifs.” Immunogenetics 50.3-4 (1999): 213-219; Duan, Fei, et al. “Genomic and bioinformatic profiling of mutational neoepitopes reveals new rules to predict anticancer immunogenicity. ” Journal of Experimental Medicine 211.11 (2014): 2231-2248; Zhang, Guang Lan, et al. “MULTIPRED: a computational system for prediction of promiscuous HLA binding peptides.” Nucleic acids research 33.suppl_2 (2005): W172-W179.; Schubert, Benjamin, et al. “EpiToolKit—a web-based workbench for vaccine design.” Bioinformatics 31.13 (2015): 2211-2213.), MHC associated peptidome identified by mass spectrometry (MS) (Abelin, Jennifer G., et al. “Mass spectrometry profiling of HLA-associated peptidomes in mono-allelic cells enables more accurate epitope prediction.” Immunity 46.2 (2017): 315-326.; Bassani-Sternberg, Michal, and George Coukos. “Mass spectrometry-based antigen discovery for cancer immunotherapy.” Current opinion in immunology 41 (2016): 9-17.; Hunt, Donald F., et al. “Characterization of peptides bound to the class I MEW molecule HLA-A2. 1 by mass spectrometry.” Science 255.5049 (1992): 1261-1263.). In some embodiments, immunodominant peptides may be predicted by referring to some parameters, such as (3-turn occurrence, hydrophilicity, surface probability, and flexibility, which have been shown to be indicative of potentially antigenic regions.

Methods of subretinal delivery are known in the art. For example, see WO 2009/105690, incorporated herein by reference. Generally, the vector and the at least one immunodominant peptide can be delivered in the form of a composition injected intraocularly (subretinally) under direct observation using an operating microscope. In some embodiments, the composition that contain the vector and the at least one immunodominant peptide is directly injected into the subretinal space outside the central retina, by utilizing a cannula of the appropriate bore size, thus creating a bleb in the subretinal space. In some embodiments, the subretinal injection of the composition is preceded by subretinal injection of a small volume (e.g., about 0.1 to about 0.5 ml) of an appropriate fluid (such as saline or Ringer's solution) into the subretinal space outside the central retina. This initial injection into the subretinal space establishes an initial fluid bleb within the subretinal space, causing localized retinal detachment at the location of the initial bleb. This initial fluid bleb can facilitate targeted delivery of the composition to the subretinal space and minimize possible administration of the composition into the choroid and the possibility of injection or reflux into the vitreous cavity. In some embodiments, this initial fluid bleb can be further injected with fluids comprising one or more compositions and/or one or more additional therapeutic agents by administration of these fluids directly to the initial fluid bleb with either the same or additional fine bore cannulas. In some embodiments of the invention, the volume of the composition injected to the subretinal space of the retina is more than about any one of 1 μl, 2 μl, 3 μl, 4 μl, 5 μl, 6 μl, 7 μl, 8 μl, 9 μl, 10 μl, 15 μl, 20 μl, 25 μl, 50 μl, 75 μl, 100 μl, 200 μl, 300 μl, 400 μl, 500 μl, 600 μl, 700 μl, 800 μl, 900 μl, or 1 mL, or any amount therebetween. One or multiple (e.g., 2, 3, or more) blebs can be created. Generally, the total volume of bleb or blebs created cannot exceed the fluid volume of the eye, for example about 4 ml in a typical human subject. The total volume of each individual bleb can be at least about 0.3 ml, or at least about 0.5 ml in order to facilitate a retinal detachment of sufficient size to expose the cell types of the central retina and create a bleb of sufficient dependency for optimal manipulation. One of ordinary skill in the art will appreciate that in creating the bleb according to the methods of the invention that the appropriate intraocular pressure must be maintained in order to avoid damage to the ocular structures.

The doses of vectors may be easily adapted by the skilled artisan, e.g., depending on the retinal disease to be treated, the subject (for example, according to his weight, metabolism, etc.), the treatment schedule, etc. A preferred effective dose within the context of this invention is a dose allowing an optimal transduction of retinal cells. Typically, from 10⁸ to 10¹² viral genomes (transducing units) are administered per dose in mice, preferably from about 10⁹ to 10¹¹. Typically, the doses of AAV vectors to be administered in humans may range from 10⁸ to 10¹² viral genomes, most preferably from 10⁹ to 10¹¹.

Pharmaceutical Compositions:

The present invention also provides a pharmaceutical composition comprising a vector containing the transgene of interest, at least one peptide that derives from the transgene product or vector and a pharmaceutically acceptable carrier, diluent, excipient, or buffer.

According to the invention, the pharmaceutical composition is compatible for subretinal injection. In some embodiments, the pharmaceutically acceptable carrier, diluent, excipient, or buffer is suitable for use in a human. Such excipients, carriers, diluents, and buffers include any pharmaceutical agent that can be administered without undue toxicity. Carriers might include cationic lipids, non-ionic lipids and polyethylene glycol (PEG) as synthetic vectors to enhance siRNA delivery. siRNA might be contained in the hydrophilic interior of the particle or polyethyleneimine and derivatives can be used to fabricate both linear and branched polymeric delivery agents. Cationic polymers with a linear or branched structure can serve as efficient transfection agents because of their ability to bind and condense nucleic acids into stabilized nanoparticles. Such materials have also been shown to stimulate nonspecific endocytosis as well as endosomal escape necessary to enhance nucleic acid uptakePharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

The invention will be further illustrated by the following figures and examples. However, these examples and figures should not be interpreted in any way as limiting the scope of the present invention.

### FIGURES

FIG. 1. Correlation Analysis Between Ocular Transgene Expression Levels and Peripheral Anti-Transgene T-Cell Immune Response in Wild Type C57BL/6 Mice.

PBS, HY peptides, or different doses (4.10⁸ to 10¹¹ vg) of AAV8-Luc2-HY were injected in the subretinal (SR) space of C57BL/6 female mice at day 0. Two weeks later, the immune response was challenged by subcutaneous immunization (SC) of either PBS:CFA or HY:CFA. The immune response of total splenocytes re-stimulated in vitro by HY peptides was assessed 1 week after immunization by IFN-γ ELISpot. In parallel, bioluminescent imaging every 3-4 days monitored the transgene expression level (5 mice/group). (A) AAV dose-dependent quantification of transgene expression by bioluminescence in the periphery at day 20. (B) Kinetic study of the loco-regional transgene expression by bioluminescence. (C) Correlation between ocular transgene expression level at day 20 and IFN-γ secretion at day 21 after in vitro anti-HY T-cell stimulation.

FIG. 2. Inhibition of Peripheral Anti-Transgene T-Cell Pro-Inflammatory Immune Response By a Subretinal Co-Injection of HY Peptides and Different Doses of AAV8 in Wild Type C57BL/6 Mice.

PBS, HY peptides, and two doses (2.10⁹ or 5.10¹⁰ vg) of AAV8-GFP-HY or AAV8-GFP-HY+HY peptides were injected in the subretinal space of C57BL/6 female mice atday 0. Two weeks later, the immune response was challenged by subcutaneous immunization of either PBS:CFA or HY:CFA. The immune response of total splenocytes re-stimulated in vitro by HY peptides was assessed 1 week after immunization by IFN-γ ELISpot. The number of spot-forming units (SFUs) from mice receiving PBS in the eye and immunized with HY peptides (positive control of anti-HY immune response) was indexed to 100 and SFUs for other mice were proportionally calculated. Bars correspond to mean+/−SEM. Data were obtained from 9 independent experiments.

FIG. 3. Inhibition of In Vivo Anti-Transgene Cytotoxicity By a Subretinal Co-Injection of HY Peptides and a High Dose of AAV8 in Wild Type C57BL/6 Mice.

PBS, a high dose (5.10¹⁰ vg) of AAV8-GFP-HY alone, or AAV8-GFP-HY+HY peptides were injected in the subretinal space of C57BL/6 female mice at day 0. Two weeks later, the immune response was challenged by subcutaneous immunization with HY:CFA. At day 17, a mixture of 3.10⁶ CD45.1⁺ CD45.2⁻CTV^{low} male and 3.10⁶ CD45.1⁻CD45.2⁺CTV^{high} female spleen cells from C57BL/6 wild type mice were injected intravenously. At day 20, blood was harvested and leucocytes were stained for flow cytometry with an anti-CD45.1-PE mAb to analyse the male cell survival in vivo. Data were obtained from 1 experiment. CTV: Cell Trace Violet.

FIG. 4. Inhibition of Peripheral Anti-AAV8 T-Cell Immune Response By a Subretinal Co-Injection of HY Peptides and High Dose of AAV8 in Wild Type C57BL/6 Mice.

PBS (Neg ctrl), HY peptides, and 5.10¹⁰ vg of AAV8-GFP-HY or AAV8-GFP-HY+HY peptides were injected in the subretinal space of C57BL/6 female mice at day 0. Two weeks later, the immune response was challenged by subcutaneous immunization of either PBS:CFA (Neg ctrl) or HY:CFA. The immune response of total splenocytes re-stimulated in vitro by AAV8 capsids was assessed 1 week after immunization by IFN-γ ELISpot and displayed as the number of spot-forming units (SFUs) per well. Data were obtained from 1 experiment.

FIG. 5. Inhibition of Peripheral Anti-Transgene T-Cell Immune Response By a Subretinal Co-Injection of HY Peptides and Different Doses of AAV8 in rd10 Mice.

PBS, HY peptides, and two doses (2.10⁹ or 5.10¹⁰ vg) of AAV8-GFP-HY or AAV8-GFP-HY+HY peptides were injected in the subretinal space of rd10 female mice at day 0. Two weeks later, the immune response was challenged by subcutaneous immunization of either PBS:CFA or HY:CFA. The immune response of total splenocytes re-stimulated in vitro by HY peptides was assessed 1 week after immunization by IFN-γ ELISpot. The number of spot-forming units (SFUs) from mice receiving PBS in the eye and immunized with HY peptides (positive control of anti-HY immune response) was indexed to 100 and SFUs for other mice were proportionally calculated. Bars correspond to mean+/−SEM. Data were obtained from 5 independent experiments.

### EXAMPLE 1

Materials & Methods

Animals

Wild-type six- to eight-week-old C57BL/6 female mice (H-2^{b}) were purchased from Charles River Laboratories (L'Arbresle, France). Animals were anesthetized either by intraperitoneal injection of 120 mg/kg ketamine (Virbac, Carros, France) and 6 mg/kg xylazine (Bayer, Lyon, France) or by inhalation of isoflurane (Baxter, Guyancourt, France). They were euthanized by cervical elongation. All mice were housed, cared for, and handled in accordance with the European Union guidelines and with the approval of the local research ethics committee (CEEA-51 Ethics Committee in Animal Experimentation, Evry, France; authorization number 2015102117539948).

AAV Vectors

AAV8-PGK-GFP-HY was produced by INSERM unit U1089 in Nantes, France. They used the tri-transfection technique in 293T cells cultured in CF10. AAV8-PGK-Luc2-HY was produced by Vector Core in Généthon, Evry, France. They used the tri-transfection technique in 293T cells cultured in roller bottles (Liu et al., 2003). Endotoxin levels were below 6 E.U/mL.

Peptides

The DEAD Box polypeptide 3 Y-linked (DBY) and Ubiquitously Transcribed tetratricopeptide repeat gene Y-linked (UTY) peptides, NAGFNSNRANSSRSS and WMHHNMDLI respectively, were synthesized by Genepep (Montpellier, France) and shown to be more than 95% pure.

Subretinal Injections

The eye was protruded under microscopic visualization and perforated with a 27G bevelled needle. A blunt 32G needle set on a 10 μL Hamilton syringe was inserted in the hole and 2 μL of PBS or UTY+DBY and/or AAV vector was injected into the subretinal space. The quality of the injection was verified by checking the detachment of the retina.

Subcutaneous Injections

PBS or UTY+DBY were emulsified in Complete Freund's Adjuvant (Sigma, Lyon, France) at a 1:1 ratio, and 100 μL of the preparation (200 μg of UTY+DBY/mouse) was injected at the base of the tail.

Cell Extraction From Spleen

After euthanasia, spleens were removed and crushed with a syringe plunger on a 70-μm filter in 2 mL of RPMI medium. Red blood cells were lysed by adding ACK buffer (8.29 g/L NH4Cl, 0.037 g/L EDTA, and 1 g/L KHCO3) for one min. Lysis was stopped by addition of complete RPMI medium (10% FBS, 1% penicillin/streptomycin, 1% glutamine, and 50 μM β-mercaptoethanol). After centrifugation, cells were counted, and the concentration was adjusted in complete RPMI medium.

Inguinal lymph nodes were crushed with a syringe plunger in 2 mL of RPMI medium. Debris were eliminated by transferring the supernatants into new tubes. After centrifugation, cells were counted and the concentration was adjusted in complete RPMI medium.

ELISpot Assay

IFN-γ Enzyme-Linked Immunospot plates (MAHAS45, Millipore, Molsheim, France) were coated with anti-IFN-γ antibody (eBiosciences, San Diego, Calif.) overnight at +4° C. Stimulation media (complete RPMI, UTY (2 μg/mL), DBY (2 μg/mL), UTY+DBY (2 μg/mL) or Concanavalin A (Sigma, Lyon, France) (5 μg/mL) were plated and 5.10⁵ splenocytes/well were added. After 24 hours of culture at +37° C., plates were washed and the secretion of IFN-γ was revealed with a biotinylated anti-IFN-γ antibody (eBiosciences), Streptavidin-Alcalin Phosphatase (Roche Diagnostics, Mannheim, Germany), and BCIP/NBT (Mabtech, Les Ulis, France). Spots were counted with an AID ELISpot iSpot Reader system ILF05 and AID ELISpot Reader v6.0 software.

Bioluminescence Imaging

Mice were injected intraperitoneally with luciferin (250 mg/kg of mice) and anesthetized with isoflurane for imaging. Ten minutes after luciferin injection, mice were placed in the imager for measurements. The imaging process used IVIS Lumina equipment and Living Image software.

In Vivo Cell Cytotoxicity Assay

Spleen cells from CD45.1⁺ CD45.2⁻ male and CD45.1⁻ CD45.2⁺ female C57BL/6 wild type mice were harvested as described above, and stained with Cell Trace Violet cell proliferation kit (Molecular Probes) in PBS at different concentration: 2 μM for male and 20 μM for female cells for 20 min at 37° C. in the dark. The reaction was quenched by addition of cold complete RPMI medium containing 10% FBS. Cells were incubated for 5 min in complete RPMI medium at 37° C. and then washed with PBS 1×. A mixture of 3.10⁶ male cells and the same number of female cells in 200 μL was injected intravenously in the experimented (CD45.1⁻CD45.2⁺) female C57BL/6 mice at day 17 of the protocol. Three days after injection, blood was harvested, red blood cells were lysed by adding ACK buffer, washed in PBS 1×, and leucocytes were stained for flow cytometry. First, cells were resuspended in 50 μL of Fc block solution (Pharmingen, BD Biosciences) diluted to 1.7 μg/mL in PBS containing 1% BSA and incubated for 10 min at 4° C. Next, 50 μL of anti-CD45.1-PE (Pharmingen, BD Biosciences) at 5 μg/mL in PBS 1% BSA was added. The cells were then incubated for 20 min at 4° C. As a control, some cells were stained in the same conditions with a the corresponding isotype antibody: mouse IgG2a,κ-PE (Pharmingen, BD Biosciences). Data were acquired on a CytoFLEX LX flow cytometer (Beckman Coulter) and analyzed with the CytExpert software (Beckman Coulter).

Statistical Analysis

Statistical analyses were performed with GraphPad Prism V6.0. After ANOVA, Tukey's test was performed. P-value<0.05: *, <0.01: **, <0.001: ***, <0.0001: ****.

Results

High Doses of Subretinal AAV8 Vectors Induce Anti-Transgene Proinflammatory T-Cell Immune Responses

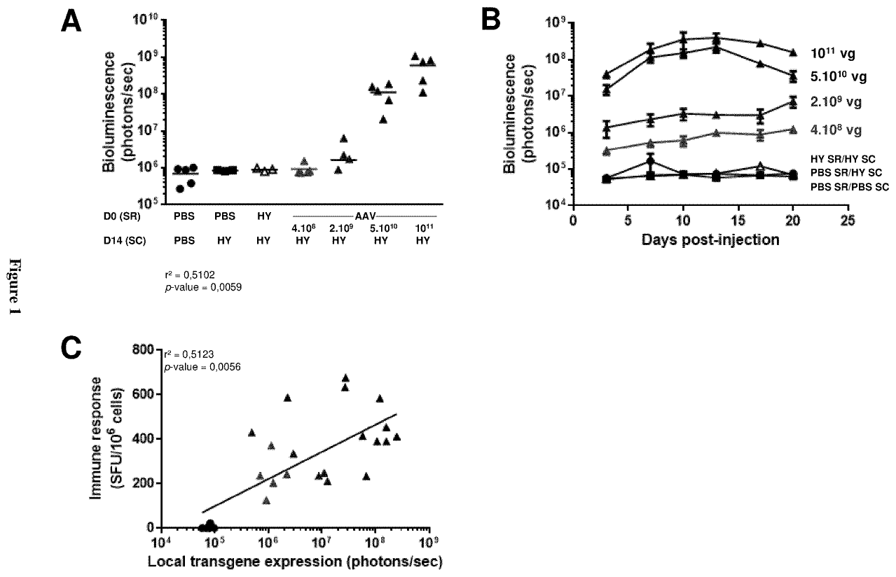

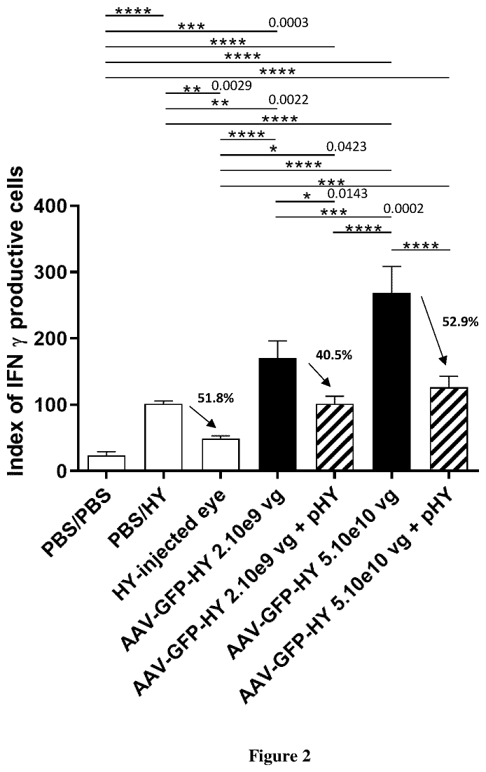

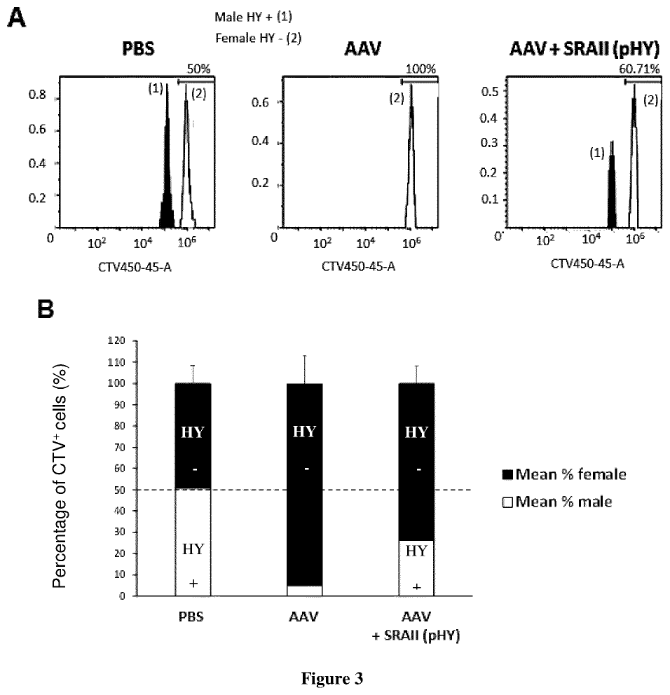

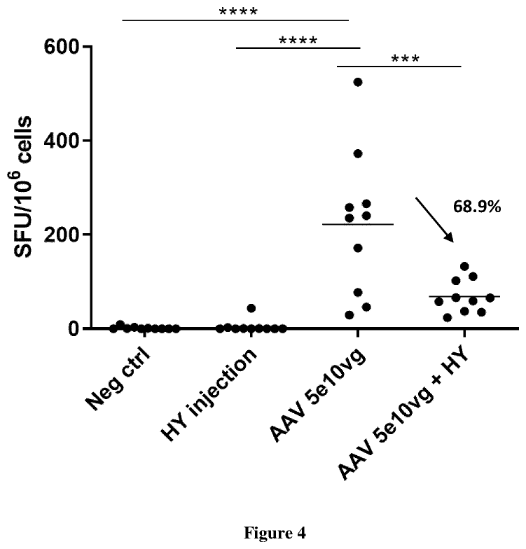

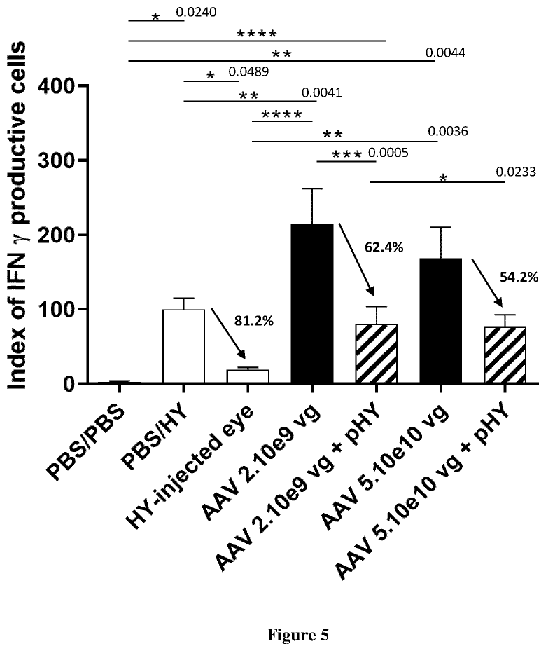

To evaluate the possibility that subretinal injection of AAV8 induces anti-transgene cellular immune responses, wild-type mice were injected with PBS, UTY+DBY (HY) peptides, or different doses of AAV8 encoding for GFP fused with HY peptides. Two weeks later, these mice were subcutaneously immunized with PBS or HY peptides. Spleen cells were harvested on day 21 and stimulated in vitro with HY peptides for ELISpot quantification of IFN-γ secretion by HY-specific T cells (FIG. 1). The challenge on day 14 makes it possible to observe the induction of subclinical immune responses or immune inhibition (Vendomèle et al., 2018).

As a positive control for anti-HY immune response, mice received PBS in the subretinal space on day 0 and HY peptides subcutaneously on day 14. In this case, 150 to 250 spot forming units (SFU) were counted in response to HY peptides, corresponding to IFN-γ-secreting spleen cells. To normalize the data from the different experiments, the index of IFN-γ secretion of the positive control was set to 100 (FIG. 1, black line). As a negative control (not shown), some mice received PBS in the subretinal space, and the immune response was challenged by subcutaneous immunization by PBS:CFA. No significant IFN-γ secretion was detected in this group (25 SFUs/10⁶ cells). We have previously reported (Vendomèle et al., 2018) that subretinal injection of HY peptides induces inhibition of T-cell immune responses (proliferation, polarization, and cytokine secretion). Thus, we used the injection of HY peptides in the subretinal space on day 0 followed by an immunization with the same peptides on day 14 as a control for immune modulation: the IFN-γ secretion index for these mice was inhibited by 65% (+/−13%) compared to the positive control. We next assessed the capacity of a wide range of AAV8-PGK-GFP-HY doses to induce an anti-transgene immune response. Low and medium doses of AAV (10³ to 2.10⁹ vg) induced levels of IFN-γ secretion similar to that of the positive control. High doses of AAV (10¹⁰ to 5.10¹⁰ vg), however, induced a two-fold increase of IFN-γ secretion compared to the positive control. Taken together, these data show that low and medium doses of AAV8 injected in the subretinal space neither induced immune modulation nor increased Th1 immune response to the transgene product. Conversely, high subretinally-injected doses of AAV8 (10¹⁰ to 5.10¹⁰ vg) induced an anti-transgene proinflammatory T-cell immune response in the periphery.

Peripheral Anti-Transgene T-Cell Immune Response is Closely Correlated with Loco-Regional Transgene Expression Levels

After demonstrating that subretinal injection of a high dose of AAV8 induced peripheral T-cell immune responses to the transgene product, we assessed the impact of the transgene expression level on the anti-transgene immune response. Mice were injected with PBS, HY peptides, or different doses of AAV8 encoding for Luciferase (Luc2) fused with HY peptides; two weeks later, they were subcutaneously immunized with PBS or HY peptides. Spleen cells were harvested on day 21 and stimulated in vitro with HY peptides to quantify IFN-γ secretion by HY-specific T cells with ELISpot. In parallel, bioluminescence imaging of the mice every three days enabled detection of Luc2 expression. We quantified transgene (Luc2) expression by the luminoscore method described elsewhere (Cosette et al., 2016). For each mouse, dorsal and ventral views were acquired, and for each view, 2 regions of interest (ROI) drawn. Local-regional (head of each mouse) transgene expression was calculated as: Head^{dorsal view}+Head^{ventral view} (blue ROIs) whereas peripheral transgene expression was calculated as: (Body^{dorsal view}+Body^{ventral view})−(Head^{dorsal view}+Head^{ventral view}) (red ROI−blue ROIs).

Control mice (negative, positive, and HY-injected) were imaged but obviously no Luc2 expression was detected locally. Medium (4.10⁸ to 2.10⁹ vg) and high (5.10¹⁰ to 10¹¹ vg) doses of AAV8 induced dose-dependent transgene expression from 3 days post-injection; this expression remained stable over 3 weeks (FIG. 1A). High doses of AAV8 induced transgene expression from 3 days that increased until day 13 and then declined until day 20 (p-value <0.01 between day 13 and day 20) (FIG. 1B). Note that the local-regional expression of the transgene was restricted to the eye, and there was no evidence of expression in the ipsilateral cervical lymph node through 21 days, regardless of the AAV dose. On day 21, an IFN-γ ELISpot assay was performed on spleen cells stimulated in vitro with HY peptides. FIG. 1C shows a plot of each mouse according to its transgene expression level on day 20 and the number of its SFUs (ELISpot). Results show that the IFN-γ secretion was correlated with local-regional (head) transgene expression (p-value=0.0056). Nonetheless, according to the coefficient of determination (r²=0.5123), this transgene expression in the eye explains only 51% of the immune response (FIG. 1C). Taken together, these data show that the transgene expression level in the eye was tightly correlated to the dose of AAV8 injected subretinally, and to the systemic anti-transgene immune response.

Subretinal-Associated Immune Inhibition Can Be Induced By a Simultaneous Injection of Peptides From the Transgene Product and AAV8 in the Retina, Even With High Doses of AAV

We have shown that subretinal injection of high doses of AAV induces proinflammatory anti-transgene immune responses that are not observed with low or medium doses. We have previously pointed out that the subretinal injection of HY peptides leads to peripheral immune inhibition (Vendomèle et al., 2018). Accordingly, we tested the possibility of using this mechanism as an immune-modulatory tool in subretinal AAV gene transfer, by co-injecting peptides from the transgene together with the AAV. Mice were injected with PBS, HY peptides, 2.10⁹ or 5.10¹⁰ vg of AAV8-PGK-GFP-HY or the same AAV8 doses plus HY. Two weeks later, mice were subcutaneously immunized with PBS or HY peptides. Spleen cells were harvested on day 21 and stimulated in vitro with HY peptides to quantify IFN-γ secretion by HY-specific T-cells by IFN-γ ELISpot assay (FIG. 2). Our results show that IFNγ secretion was inhibited by 40.5% in the mice that received HY peptides subretinally, compared with the positive control. Interestingly, co-injecting HY peptides with 2.10⁹ vg of AAV8, compared to the 2.10⁹ vg of AAV8 alone, reduced IFN-γ secretion significantly (p=0.0007), by half In the same way, co-injection of HY peptides with the high dose of AAV8 decreased by 52.9% IFN-γ secretion by HY-specific T cells. Taken together, these data show that co-injection of immunodominant peptides from the transgene together with different doses of AAV8 inhibited the T-cell pro-inflammatory cytokine secretion in response to the transgene.

Anti-Transgene Cell Cytotoxicity Can Be Inhibited By a Simultaneous Injection of Peptides From the Transgene Product and AAV8 in the Retina

Since we have demonstrated that a simultaneous subretinal injection of high doses of AAV and peptides from the transgene product can lead to peripheral inhibition of the secretion by T-cells of pro-inflammatory cytokines such as IFNγ, we investigated the potentiality to inhibit the anti-transgene in vivo cytotoxicity. PBS, a high dose (5.10¹⁰ vg) of AAV8-GFP-HY alone, or AAV8-GFP-HY+HY peptides were injected in the subretinal space of C57BL/6 female mice on day 0, and two weeks later the immune response was challenged by subcutaneous immunization with HY:CFA. At day 17, a mixture of 3.10⁶ CD45.1⁺ CD45.2⁻ CTV^{low} male and 3.10⁶ CD45.1⁻ CD45.2⁺ CTV^{high} female spleen cells from C57BL/6 wild type mice were injected intravenously. At day 20, leucocyte analysis showed that the same proportion of male HY⁺ (CTV^{low}) and female HY⁻ (CTV^{high}) cells survived in the PBS-injected control group (FIG. 3A, 3B). As expected, very few male cells survived in the AAV-GF-HY injected group (5.2% male vs 94.8 female cells), in contrast to the AAV+HY peptides immunomodulatory group (26.4% male vs 73.6 female cells). Thus, co-injection of immunodominant peptides from the transgene together with a high dose of AAV8 is able to inhibit in vivo anti-transgene cell cytotoxicity.

A Bystander Inhibition of Peripheral AAV8 Capsid T-Cell Immune Responses Can Be Obtained By a Simultaneous Injection of Peptides From the Transgene Product and AAV8 in the Retina

Since a subretinal injection of high doses of AAV and peptides from the transgene product can lead to peripheral inhibition of the pro-inflammatory cytokine secretion by T-cells and anti-transgene in vivo cytotoxicity, we wondered whether SRAII could also affect anti-capsid specific T-cell immune responses that are usually triggered by an AAV injection. For this purpose, PBS (negative control group), HY peptides (SRAII control group), and 5.10¹⁰ vg of AAV8-GFP-HY, or AAV8-GFP-HY+HY peptides were injected in the subretinal space of C57BL/6 female mice at day 0. Two weeks later, the immune response was challenged by subcutaneous immunization of either PBS:CFA (negative control group) or HY:CFA. The T-cell immune response was assessed by in vitro re-stimulation with AAV8 capsids 1 week after immunization by IFN-γ ELISpot assay (FIG. 4). Our results show that IFNγ secretion was inhibited by 68.9% in mice receiving AAV8+HY peptides subretinally, compared with mice injected with AAV8 alone. Interestingly, since the AAV8 capsid did not contain HY peptides, it indicates a bystander immunosuppression directed against the anti-capsid (AAV8) T-cell responses that were generated simultaneously with the anti-transgene (HY) specific T-cell activation. Hence, these data show that co-injection of immunodominant peptides from the transgene together with high doses of AAV8 can also inhibit anti-capsid specific T-cell pro-inflammatory cytokine secretion.

Discussion:

AAV-mediated gene transfer in the retina has advanced enormously over the past 20 years, from the proof-of-concept in 1996 (Ali et al., 1996) to clinical trials in the 2000s. Despite initially promising results, long-term follow-up in some clinical trials have revealed a secondary loss of vision after the initial AAV-induced improvement (Bainbridge et al., 2015; Jacobson et al., 2015). This led us to explore the possibility of subclinical anti-transgene immune response. Actually patients enrolled in clinical trials received immunosuppressive treatments, either locally and/or systemically during the first few days after AAV injection, which probably limited, delayed, or masked the induction of immune responses. Because transgene expression continues to be expressed after the treatment, however, an immune response to the transgene product can be induced over the long term.

Several studies have highlighted the immune-privileged status of the eye. Delayed-type hypersensitivity measurements have shown that subretinal injection of ovalbumin induces inhibition of the Thl profile in the periphery (Wenkel and Streilein, 1998), and McPherson et al. showed that regulatory T cells specific to retinal antigens are generated there (McPherson et al., 2011). We further characterized systemic immune responses associated with the subretinal space and showed that subretinal injection of HY, a male antigen, induced SRAII, that inhibited the proliferation and polarization of T cells,) (Vendomele et al., 2018).

Since the antigenic load is closely correlated with immune responses, as vaccine or AAV vector studies have shown (Gu et al., 2018; Khabou et al., 2018), and since immune responses are likely to be dependent on the AAV dose (Ramachandran et al., 2016), we wondered whether they are also correlated with the transgene expression level. Bioluminescence imaging revealed dose-dependent transgene expression in the eye. It is nonetheless important to bear in mind that transgene peptides might also be processed by retinal antigen-presenting cells (such as microglia) that could then migrate to the periphery (e.g., spleen, cervical lymph nodes) and trigger a systemic anti-transgene immune response. A study of local immune responses and retinal structure would now be of major interest, to determine the existence of an association between the transgene expression level and immune response in the eye and in the periphery. Furthermore, the patients in ocular AAV-mediated clinical trials received local and/or systemic immunosuppressive treatments (e.g., prednisolone) before and for a few days after their injection. This kind of approach enables non-specific inhibition of the immune response, which can be deleterious for the patient. Moreover, its effect is only transient, while the transgene is expressed over the long term. We therefore sought to partially inhibit the anti-transgene and the anti-capsid immune responses induced in our context by exploiting the SRAII mechanism. The co-injection of immunodominant peptides from the transgene with medium doses of AAV8 induced both the inhibition of the immune response to the transgene product and the AAV capsid. The role of transgene- and bystander capsid-specific modulation that we began to study by co-injection of immunodominant peptides from the transgene should be examined in greater depth in further studies.

All the experiments in our study were performed in wild-type C57BL/6 female mice, which enabled us to highlight and decipher the subclinical immune mechanisms involved in AAV-mediated ocular gene transfer. A useful question to be further examined for gene therapy applications is how these mechanisms would be influenced by the presence of various ocular pathologies. Several ocular pathologies affect the blood-retinal barrier (Milam et al., 1998; Vinores et al., 1995; Wang et al., 2011) and the local environment is inflammatory (Chen and Xu, 2015; Yoshida et al., 2013). In particular, the use of retinal degeneration models such as rd10 mice would enable new insights. Although we might hypothesize that proinflammatory immune responses would also be induced in that context, their potential for modulation by co-injection is uncertain. The possibility of inducing the SRAII mechanism in a context of retinal degeneration should be explored (cf Example 2 below).

Over the long term, these results could lead to improvement in the safety and effectiveness of AAV-mediated gene transfer for patients. Our work opens a new avenue of investigation in the field of immune responses in AAV-mediated subretinal gene transfer, and may provide insights for transgene- and capsid-specific immune modulation in a larger context.

### EXAMPLE 2

To confirm the relevance of the present claim in a pathophysiological context, experiments were done in the rd10 murine model of retinal degeneration, aiming to prevent induction of T-cell immune responses to the transgene product after ocular gene transfer. The retinal degeneration 10 (rd10) murine model is characterized by is a spontaneous missense point mutation in Pde6b (cGMP phosphodiesterase 6B, rod receptor, beta polypeptide) gene. The rd10 phenotype has a late onset and mild retinal degeneration and provide a good experimental drug therapy model for retinitis pigmentosa.

Different doses (2.10⁹ or 5.10¹⁰ vg) of AAV8 encoding the GFP reporter protein fused with the HY male antigen, under PGK promoter, were injected at day 0 into the subretinal space of adult immunocompetent rd10 female mice. The mice were subcutaneously immunized at day 14 with or without HY peptides, and their T-cell immune responses in the spleen were analyzed at day 21 by an IFN-γ ELISpot assay after in vitro restimulation with HY peptides. Data showed that subretinal injection of AAV8 induced an anti-transgene proinflammatory T-cell immune response (FIG. 5). Subretinal co-injection at day 0 with AAV8 and HY peptides leaded to a modulation (at least 50% inhibition) of the anti-transgene T-cell immune response, even at high dose of vector (5.10¹⁰ vg) (FIG. 5).

Taken together, these data confirm that a subretinal co-injection of a vector and peptides of the transgene product can counteract the proinflammatory peripheral immune responses to the transgene induced by the AAV introduction in the eye, even in pathophysiological conditions.

### REFERENCES

Throughout this application, various references describe the state of the art to which this invention pertains. The disclosures of these references are hereby incorporated by reference into the present disclosure.

Acland, G. M., Aguirre, G. D., Ray, J., Zhang, Q., Aleman, T. S., Cideciyan, A. V., Pearce-Kelling, S. E., Anand, V., Zeng, Y., Maguire, A. M., et al. (2001). Gene therapy restores vision in a canine model of childhood blindness. Nat. Genet. 28, 92-95.

Acland, G. M., Aguirre, G. D., Bennett, J., Aleman, T. S., Cideciyan, A. V., Bennicelli, J., Dejneka, N. S., Pearce-Kelling, S. E., Maguire, A. M., Palczewski, K., et al. (2005). Long-Term Restoration of Rod and Cone Vision by Single Dose rAAV-Mediated Gene Transfer to the Retina in a Canine Model of Childhood Blindness. Mol. Ther. 12, 1072-1082.

Ali, R. R., Reichel, M. B., Thrasher, A. J., Levinsky, R. J., Kinnon, C., Kanuga, N., Hunt, D. M., and Bhattacharya, S. S. (1996). Gene Transfer into the Mouse Retina Mediated by an Adeno-Associated Viral Vector. Hum. Mol. Genet. 5, 591-594.

Allocca, M., Mussolino, C., Garcia-Hoyos, M., Sanges, D., Iodice, C., Petrillo, M., Vandenberghe, L. H., Wilson, J. M., Marigo, V., Surace, E. M., et al. (2007). Novel adeno-associated virus serotypes efficiently transduce murine photoreceptors. J. Virol. 81, 11372-11380.

Auricchio, A., Kobinger, G., Anand, V., Hildinger, M., O'Connor, E., Maguire, A. M., Wilson, J. M., and Bennett, J. (2001). Exchange of surface proteins impacts on viral vector cellular specificity and transduction characteristics: the retina as a model. Hum. Mol. Genet. 10, 3075-3081.

Bainbridge, J. W. B., Mehat, M. S., Sundaram, V., Robbie, S. J., Barker, S. E., Ripamonti, C., Georgiadis, A., Mowat, F. M., Beattie, S. G., Gardner, P. J., et al. (2015). Long-Term Effect of Gene Therapy on Leber's Congenital Amaurosis. N. Engl. J. Med. 372, 1887-1897.

Chan, N. S.-W., Chee, S.-P., Caspers, L., and Bodaghi, B. (2017). Review for Disease of the Year: Clinic of Cytomegalovirus-Induced Anterior Uveitis. Ocul. Immunol. Inflamm. 1-9.

Chen, M., and Xu, H. (2015). Parainflammation, chronic inflammation, and age-related macular degeneration. J. Leukoc. Biol. 98, 713-725.

Cideciyan, A. V., Jacobson, S. G., Beltran, W. A., Sumaroka, A., Swider, M., Iwabe, S., Roman, A. J., Olivares, M. B., Schwartz, S. B., Komaromy, A. M., et al. (2013). Human retinal gene therapy for Leber congenital amaurosis shows advancing retinal degeneration despite enduring visual improvement. Proc. Natl. Acad. Sci. 110, E517-E525.

Cosette, J., Ben Abdelwahed, R., Donnou-Triffault, S., Sautès-Fridman, C., Flaud, P., and Fisson, S. (2016). Bioluminescence-Based Tumor Quantification Method for Monitoring Tumor Progression and Treatment Effects in Mouse Lymphoma Models. J. Vis. Exp. JoVE.

Dupont, J.-B., Tournaire, B., Georger, C., Marolleau, B., Jeanson-Leh, L., Ledevin, M., Lindenbaum, P., Lecomte, E., Cogne, B., Dubreil, L., et al. (2015). Short-lived recombinant adeno-associated virus transgene expression in dystrophic muscle is associated with oxidative damage to transgene mRNA. Mol. Ther. Methods Clin. Dev. 2, 15010.

Gu, H., Gao, Y., Zhou, S., Sun, F., Zhao, Z., Wang, K., Zhao, L., Zhang, P., Wang, Z., Zhang, S., et al. (2018). Bivalent vaccine platform based on ca influenza virus vaccine elicits protective immunity against human adenoviruses. Antiviral Res.

Hauswirth, W. W., Aleman, T. S., Kaushal, S., Cideciyan, A. V., Schwartz, S. B., Wang, L., Conlon, T. J., Boye, S. L., Flotte, T. R., Byrne, B. J., et al. (2008). Treatment of Leber Congenital Amaurosis Due to RPE65 Mutations by Ocular Subretinal Injection of Adeno-Associated Virus Gene Vector: Short-Term Results of a Phase I Trial. Hum. Gene Ther. 19, 979-990.

Jacobson, S. G., Boye, S. L., Aleman, T. S., Conlon, T. J., Zeiss, C. J., Roman, A. J., Cideciyan, A. V., Schwartz, S. B., Komaromy, A. M., Doobrajh, M., et al. (2006a). Safety in nonhuman primates of ocular AAV2-RPE65, a candidate treatment for blindness in Leber congenital amaurosis. Hum. Gene Ther. 17, 845-858.

Jacobson, S. G., Acland, G. M., Aguirre, G. D., Aleman, T. S., Schwartz, S. B., Cideciyan, A. V., Zeiss, C. J., Komaromy, A. M., Kaushal, S., Roman, A. J., et al. (2006b). Safety of recombinant adeno-associated virus type 2-RPE65 vector delivered by ocular subretinal injection. Mol. Ther. J. Am. Soc. Gene Ther. 13, 1074-1084.

Jacobson, S. G., Cideciyan, A. V., Roman, A. J., Sumaroka, A., Schwartz, S. B., Heon, E., and Hauswirth, W. W. (2015). Improvement and Decline in Vision with Gene Therapy in Childhood Blindness. N. Engl. J. Med. 372, 1920-1926.

Khabou, H., Cordeau, C., Pacot, L., Fisson, S., and Dalkara, D. (2018). Dosage thresholds and influence of transgene cassette in AAV-related toxicity. Hum. Gene Ther.

Kurniawan, E. D., Rocke, J. R., Sandhu, S. S., and Allen, P. J. (2017). Predictors of visual outcome and the role of early vitrectomy in streptococcal endophthalmitis. Clin. Experiment. Ophthalmol.

Le Meur, G., Stieger, K., Smith, A. J., Weber, M., Deschamps, J. Y., Nivard, D., Mendes-Madeira, A., Provost, N., Péréon, Y., Cherel, Y., et al. (2007). Restoration of vision in RPE65-deficient Briard dogs using an AAV serotype 4 vector that specifically targets the retinal pigmented epithelium. Gene Ther. 14, 292-303.

Le Meur, G., Lebranchu, P., Billaud, F., Adjali, O., Schmitt, S., Bézieau, S., Péréon, Y., Valabregue, R., Ivan, C., Darmon, C., et al. (2017). Safety and Long-Term Efficacy of AAV4 Gene Therapy in Patients with RPE65 Leber Congenital Amaurosis. Mol. Ther. J. Am. Soc. Gene Ther.

Lebherz, C., Maguire, A., Tang, W., Bennett, J., and Wilson, J. M. (2008). Novel AAV serotypes for improved ocular gene transfer. J. Gene Med. 10, 375-382.

Liu, Y. L., Wagner, K., Robinson, N., Sabatino, D., Margaritis, P., Xiao, W., and Herzog, R. W. (2003). Optimized production of high-titer recombinant adeno-associated virus in roller bottles. BioTechniques 34, 184-189.

Maclachlan, T. K., Lukason, M., Collins, M., Munger, R., Isenberger, E., Rogers, C., Malatos, S., Dufresne, E., Morris, J., Calcedo, R., et al. (2011). Preclinical safety evaluation of AAV2-sFLT01—a gene therapy for age-related macular degeneration. Mol. Ther. J. Am. Soc. Gene Ther. 19, 326-334.

Maguire, A. M., High, K. A., Auricchio, A., Wright, J. F., Pierce, E. A., Testa, F., Mingozzi, F., Bennicelli, J. L., Ying, G., Rossi, S., et al. (2009). Age-dependent effects of RPE65 gene therapy for Leber's congenital amaurosis: a phase 1 dose-escalation trial. Lancet Lond. Engl. 374, 1597.

McPherson, S. W., Heuss, N. D., Lehman, U., and Gregerson, D. S. (2011). Generation of Regulatory T Cells to Antigen Expressed in the Retina. Curr. Immunol. Rev. 7, 344-349.

Milam, A. H., Li, Z. Y., and Fariss, R. N. (1998). Histopathology of the human retina in retinitis pigmentosa. Prog. Retin. Eye Res. 17, 175-205.

Petit, L., Lheriteau, E., Weber, M., Le Meur, G., Deschamps, J.-Y., Provost, N., Mendes-Madeira, A., Libeau, L., Guihal, C., Colle, M.-A., et al. (2012). Restoration of vision in the pde6β-deficient dog, a large animal model of rod-cone dystrophy. Mol. Ther. J. Am. Soc. Gene Ther. 20, 2019-2030.

Ramachandran, P. S., Lee, V., Wei, Z., Song, J.Y., Casal, G., Cronin, T., Willett, K., Huckfeldt, R., Morgan, J. I. W., Aleman, T. S., et al. (2016). Evaluation of Dose and Safety of AAV7m8 and AAV8BP2 in the Non-Human Primate Retina. Hum. Gene Ther.

Rizzolo, L. J., Peng, S., Luo, Y., and Xiao, W. (2011). Integration of tight junctions and claudins with the barrier functions of the retinal pigment epithelium. Prog. Retin. Eye Res. 30, 296-323.

Stein-Streilein, J. (2013). Mechanisms of immune privilege in the posterior eye. Int. Rev. Immunol. 32, 42-56.

Taylor, A. W., Alard, P., Yee, D. G., and Streilein, J. W. (1997). Aqueous humor induces transforming growth factor-β (TGF-β)-producing regulatory T-cells. Curr. Eye Res. 16, 900-908.

Vandenberghe, L. H., Bell, P., Maguire, A. M., Xiao, R., Hopkins, T. B., Grant, R., Bennett, J., and Wilson, J. M. (2013). AAV9 targets cone photoreceptors in the nonhuman primate retina. PloS One 8, e53463.

Vendomèle, J., Khebizi, Q., and Fisson, S. (2017). Cellular and Molecular Mechanisms of Anterior Chamber-Associated Immune Deviation (ACAID): What We Have Learned from Knockout Mice. Front. Immunol. 8.

Vendomèle, J., Dehmani, S., Khebizi, Q., Galy, A., and Fisson, S. (2018). Subretinal injection of HY peptides induces systemic antigen-specific inhibition of effector CD4+ and CD8+ T-cell responses. Front. Immunol. 9.

Vinores, S. A., Kiichle, M., Derevjanik, N. L., Henderer, J. D., Mahlow, J., Green, W. R., and Campochiaro, P. A. (1995). Blood-retinal barrier breakdown in retinitis pigmentosa: light and electron microscopic immunolocalization. Histol. Histopathol. 10, 913-923.

Wang, Q., Song, S.-K., Zhang, H., Berkowitz, B. A., Chen, S., Wickline, S. A., and Chen, J. (2011). Photoreceptor degeneration changes magnetic resonance imaging features in a mouse model of retinitis pigmentosa. Magn. Reson. Med. 65, 1793-1798.

Weber, M., Rabinowitz, J., Provost, N., Conrath, H., Folliot, S., Briot, D., Cherel, Y., Chenuaud, P., Samulski, J., Moullier, P., et al. (2003). Recombinant adeno-associated virus serotype 4 mediates unique and exclusive long-term transduction of retinal pigmented epithelium in rat, dog, and nonhuman primate after subretinal delivery. Mol. Ther. J. Am. Soc. Gene Ther. 7, 774-781.

Wenkel, H., and Streilein, J. W. (1998). Analysis of immune deviation elicited by antigens injected into the subretinal space. Invest. Ophthalmol. Vis. Sci. 39, 1823-1834.

Yoshida, N., Ikeda, Y., Notomi, S., Ishikawa, K., Murakami, Y., Hisatomi, T., Enaida, H., and Ishibashi, T. (2013). Laboratory evidence of sustained chronic inflammatory reaction in retinitis pigmentosa. Ophthalmology 120, e5-12.

1. A method for preventing a secondary vision loss in a patient who received an ocular gene therapy with a vector containing a transgene comprising administering to the patient a therapeutically effective dose of at least one peptide that derives from a transgene product or the vector, simultaneously with gene therapy, thereby preventing induction of immune responses to the transduced cells expressing the transgene product.

2. The method of claim 1 wherein the immune response is a cellular cytotoxic response.

3. A method for expressing a transgene of interest in the retina of a patient comprising injecting into a subretinal space of the patient a therapeutically effective amount of a vector containing a transgene of interest in combination with a therapeutically effective amount of at least one peptide that derives from a product of the transgene or the vector.

4. A method of treating a retinal disease in a patient in need thereof, comprising injecting into the subretinal space of the patient an amount of a vector containing a transgene of interest in combination with a therapeutically effective amount of at least one peptide that derives from a product of the transgene or the vector.

5. The method of claim 1, to wherein the patient suffers from a retinal acquired disease that is macular degeneration or diabetic retinopathies.

6. The method of claim 1, wherein the patient suffers from an inherited retinal disease selected from the group consisting of retinitis pigmentosa, Leber's congenital amaurosis, X-linked retinoschisis, autosomal recessive severe early-onset retinal degeneration (Leber's Congenital Amaurosis), congenital achromatopsia, Stargardt's disease, Best's disease, Doyne's disease, cone dystrophy, retinitis pigmentosa, X linked retinoschisis, Usher's syndrome, age related macular degeneration, atrophic age related macular degeneration (AMD), neovascular AMD, diabetic maculopathy, proliferative diabetic retinopathy (PDR), cystoid macular oedema, central serous retinopathy, retinal detachment, intra-ocular inflammation, glaucoma, posterior uveitis, choroideremia, and Leber hereditary optic neuropathy.

7. The method of claim 1, wherein the transgene product is a polypeptide that enhances the function of a retinal cell.

8. The method of claim 1, wherein the transgene product is an endonuclease that provides site-specific knock-down of gene function.

9. The method of claim 1, wherein the vector containing the transgene is selected from the group consisting of viral and non-viral vectors.

10. The method of claim 9, wherein the vector is an adenoviral vector (AVV).

11. The method of claim 10 wherein the AAV vector is an AAV8 vector.

12. The method of claim 1, wherein the peptide is an immunodominant peptide that derives from the transgene product or vector.

13. The method of claim 12 wherein the vector is an AAV vector and the immunodominant peptide derives from a capsid protein of the AAV vector.

14. The method of claim 13 wherein the immunodominant peptide derives from the VP1, VP2, or VP3 capsid protein of the AAV vector.

15. The method of claim 12 wherein the immunodominant peptide derives from the transgene product.

16. The method of claim 1, wherein the vector is injected in the subretinal space simultaneously with 2, 3, 4, 5, 6, 8, 9 or 10 immunodominant peptides.

17. The method of claim 12 wherein the vector is injected with at least one immunodominant peptide comprising a MHC-class I restricted epitope and/or at least one immunodominant peptide comprising a MHC-class II restricted epitope.

18. A pharmaceutical composition comprising a vector containing the transgene of interest, at least one peptide that derives from the transgene product or vector and a pharmaceutically acceptable carrier, diluent, excipient, or buffer.

19. The method of claim 5, wherein the macular degeneration is age related macular degeneration.
